(19) United States
(12) Patent Application Publication
(10) Pub. No.: US 2014/0037627 A1
(43) Pub. Date: Feb. 6, 2014

(54) MODULATION OF PILR RECEPTORS TO TREAT MICROBIAL INFECTIONS

(71) Applicant: MERCK SHARP & DOHME CORP., Rahway, NJ (US)

(72) Inventors: Antara Banerjee, Fremont, CA (US); Paul G. Heyworth, San Francisco, CA (US)

(73) Assignee: MERCK SHARP & DOHME CORP., Rahway, NJ (US)

(21) Appl. No.: 13/962,828

(22) Filed: Aug. 8, 2013

Related U.S. Application Data
(63) Continuation of application No. 13/321,397, filed on Feb. 1, 2012, now abandoned.
(60) Provisional application No. 61/179,978, filed on May 20, 2009.

(51) Int. Cl. A61K 39/395; A61K 31/713

(52) U.S. Cl. CPC A61K 39/3955; A61K 31/713
USPC 424/134.1; 424/139.1; 514/2.7; 424/133.1; 514/44 A

(57) ABSTRACT

The present invention provides methods of using agonists and antagonists of PILRα and PILRβ, respectively, to treat S. aureus infection, in particular, S. aureus infections of the lungs. Also provided are use agonists and antagonists of PILRα and PILRβ, respectively, to prevent such infections.

### FIELD OF THE INVENTION

The present invention provides methods of modulating FDF03 receptors to treat microbial infections, in particular bacterial infection

### BACKGROUND OF THE INVENTION

Stahylococcus aureus has long been recognized as one of the most important bacteria that cause disease in humans. It is the leading cause of skin and soft tissue infections such as abscesses (boils), furuncles, and cellulitis. Although most staphylococcal infections are not serious, S. aureus can cause serious infections such as bloodstream infections, pneumonia, or bone and joint infections. The skin and mucous membranes are usually an effective barrier against infection. However, if these barriers are breached (e.g., skin damage due to trauma or mucosal damage due to viral infection) S. aureus may gain access to underlying tissues or the bloodstream and cause infection.

Pneumonia is defined as an acute infection of the lung parenchyma. Infection of this normally sterile environment by pathogenic bacteria results in their proliferation in the lungs and, ultimately, to bacterial invasion of the epithelial linings of the alveoli. Components of the invading bacteria induce production of proinflammatory cytokines and chemokines, including TNFα, IL-10 and IL-8, that attract and stimulate neutrophils and monocytes from the blood stream to the site of infection. Staphylococcus aureus, a Gram positive extracellular bacterium accounts for 2% of community-associated pneumonia and up to 20% nosocomial pneumonia as well as being a major cause of sepsis (see, e.g., Fournier and Philpott (2005 Clin. Micorbol. Rev. 18:521-540 and Lowy (1998) N. Engl. J. Med. 339:520-532). Recent studies have indicated a high prevalence of community acquired methicillin-resistant S. aureus (MRSA) in otherwise healthy individuals (see, e.g., Gillet et al. (2002) Lancet 359:753-759). This growing resistance of S. aureus to β-lactam antibiotics warrants the search for new therapeutics targets to combat pulmonary pneumonia caused by this pathogen.

Neutrophils, monocytes and macrophages constitute a major fraction of blood and tissue leukocytes. They are responsible for mounting a rapid innate immune response and also for initiating and directing adaptive immunity (see, e.g., Nathan (2006) Nat. Rev. Immunol. 6:173-182). Upon activation these cells migrate to sites of infection, where they phagocytose and eradicate invading pathogens through an arsenal of cytotoxic agents in preformed granules and with the release of additional inflammatory chemokines, cytokines and reactive oxygen species. They are unique in their capacity to destroy and heal injured tissue and offer potential therapeutic promise for pharmacological intervention to promote and restrain inflammation (see, e.g., Craig, et al. (2009) Infect. Immunol. 77:568-575). In the lung, the local inflammatory response to a bacterial pathogen such as S. aureus is mediated through a tight regulation and interaction between pattern recognition receptors and certain stimulatory innate immunoreceptors present on cells of the myeloid lineage (see, e.g., Underhill and Gantner (2000) Microbes Infect. 6:1368-73). Previous reports have shown that effective defense against S. aureus infection in the lung of immunocompetent mice is primarily accomplished by the host's ability to evoke a strong innate immune response through neutrophil and macrophage sequestration. However, the precise function of many immune regulatory receptors present on these cells and their involvement in the molecular and cellular mechanisms of host defense against pulmonary S. aureus infection still remains to be understood.

Neutrophils and macrophages express a number of paired immune regulatory receptors of either the C-type lectin- or Ig-superfamilies. Paired receptors have similar ectodomains and are thought to interact with the same ligand, but function to produce opposing signals (see, e.g., Ravetch and Lanier (2000) Science 290:84-89 and Lanier (2001) Curr. Opin. Immunol. 13:326-331). In order to avoid any detrimental and inappropriate inflammatory response, it is critical to preserve a fine balance between the activation and inhibitory signals. The paired immunoglobulin-type 2-like receptor (PILR) family comprises two isoforms, inhibitory PILRα (aka inhibitory FDF03) and activating PILRβ (aka activating FDF03) isoforms, and is well conserved among most mammals (see, e.g., Fournier, et al. (2000) J. Immunol. 165:1197-1209 and Shiratori, et al. (2004) J. Exp. Med. 199:525-533). These paired receptors belong to the v-type immunoglobulin superfamily and are mapped to chromosome 7q22 in human. PILRα possesses two ITIM motifs in its cytoplasmic domain and delivers inhibitory signals through recruitment of SHP-1 via its amino—terminal SH2 domain (see, e.g., Mousseau, et al. (2000) J. Biol. Chem. 275:4467-4474). Conversely, PILRβ, which does not contain an ITIM motif, associates with the adaptor molecule DAP12 through positively charged amino acid residues in the PILR transmembrane region and transduces an activating signal throughout the DAP12 immunoregulatory tyrosine-based activation motif (ITAM; see, e.g., Shiratori, et al. supra).

Both isoforms are expressed on the cell surface of neutrophils, monocytes, macrophages and dendritic cells. Additionally, PILRβ is also present on NK cells and a small population of T cells in both mouse and human (see, e.g., Fournier, et al. supra; and Shiratori, et al. supra). Initial studies reported CD99 to be a potential ligand for both receptors in mouse (see, e.g., Shiratori, et al. supra). However, more recently it was observed that the O-glycan sugar chain on CD99 is involved in receptor recognition (see, e.g., Wang, et al. (2008) J. Immunol. 180:1686-1693). Recent studies have also demonstrated glycoprotein-B of the herpes simplex virus −1 to be a ligand for PILRα (see, e.g, Satoh, et al. (2008) Cell 132:935-944), signifying an alternative route for viral entry into the infected cells.

Although, the presence of PILRα and PILRβ on myeloid cells is well known, their function in microbial infections is not well understood. Furthermore, given the increase of antibiotic resistance of various infectious agents, a need exists to develop alternative treatments that function to mediate the body's innate immunity. The present invention fills this need by providing modulators of PILRα and PILRβ that function to clear such infections.

### SUMMARY OF THE INVENTION

The present invention is based, in part, upon the discovery that modulating PILR receptors can affect bacterial infection by S. aureus.

The present invention provides a method of modulating an S. aureus infection comprising administering to a subject in need of such treatment, an effective amount of an antagonist of PILRβ. In certain embodiments, antagonist of PILRβ is an antibody, antibody fragment, or antibody conjugate, including a polyclonal antibody, a monoclonal antibody, a recombinant antibody, a humanized antibody or fragment thereof, a fully human antibody or fragment thereof. The antagonist can also be a soluble PILRβ polypeptide, or a soluble PILRβ polypeptide fused to a heterologous protein. For example, a soluble PILRβ polypeptide or fusion polypeptide may comprise residues 20-191 of SEQ ID NO: 4. The antagonist of PILRβ reduces S. aureus infection. In one embodiment the S. aureus infection is in at least one lung. The invention also provides that the antagonist of PILRβ is administered with at least one antibiotic having bateriocidal or bacteriostatic activity against S. aureus.

The present invention encompasses a method of modulating an S. aureus infection comprising administering to a subject in need of such treatment, an effective amount of an agonist of PILRα. In one embodiment, the antagonist of PILRα is an antibody, antibody fragment, or antibody conjugate, including a polyclonal antibody, a monoclonal antibody, a recombinant antibody, a humanized antibody or fragment thereof, a fully human antibody or fragment thereof. The agonist of PILRα reduces S. aureus infection. In a further embodiment the S. aureus infection is in at least one lung. The invention also provides that the agonist of PILRα is administered with at least one antibiotic having bateriocidal or bacteriostatic activity against S. aureus.

The present invention provides a method of prophylactically treating a subject against an S. aureus infection comprising administering to the subject in need of such treatment, an effective amount of an antagonist of PILRβ. In one embodiment, the antagonist of PILRβ is an antibody, antibody fragment, or antibody conjugate, including a polyclonal antibody, a monoclonal antibody, a recombinant antibody, a humanized antibody or fragment thereof, a fully human antibody or fragment thereof. The antagonist can also be a soluble PILRβ polypeptide, or a soluble PILRβ polypeptide fused to a heterologous protein. For example, a soluble PILRβ polypeptide or fusion polypeptide may comprise residues 20-191 of SEQ ID NO: 4. The antagonist of PILRβ prevents S. aureus infection. In a further embodiment, the S. aureus infection is in at least one lung. The invention also provides the antagonist of PILRβ is administered with at least one antibiotic having bateriocidal or bacteriostatic activity against S. aureus.

The present invention encompasses a method of prophylactically treating a subject against an S. aureus infection comprising administering to the subject in need of such treatment, an effective amount of an agonist of PILRα. In one embodiment, agonist of PILRα is an antibody, antibody fragment, or antibody conjugate, including a polyclonal antibody, a monoclonal antibody, a recombinant antibody, a humanized antibody or fragment thereof, a fully human antibody or fragment thereof. The agonist of PILRα prevents S. aureus infection. In a further embodiment, the S. aureus infection is in at least one lung. The invention also provides that the agonist of PILRα is administered with at least one antibiotic having bateriocidal or bacteriostatic activity against S. aureus.

In other embodiments the antagonist of PILRβ comprises a polynucleotide. In various embodiments the polynucleotide is an antisense nucleic acid (e.g. antisense RNA) or an interfering nucleic acid, such as a small interfering RNA (siRNA). In one embodiment the polynucleotide antagonist of PILRβ is delivered in gene therapy vector, such as an adenovirus, lentivirus, retrovirus or adenoassociated virus vector. In another embodiment the polynucleotide antagonist of PILRβ is delivered as a therapeutic agent.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

As used herein, the phrase “pathogenic agent” means an agent which causes a disease state or affliction in an animal. Included within this definition, for examples, are bacteria, protozoans, fungi, viruses and metazoan parasites which either produce a disease state or render an animal infected with such an organism susceptible to a disease state (e.g., a secondary infection). Further included are species and strains of the genus Staphylococcus which produce disease states in animals.

As used herein, the term “organism” means any living biological system, including viruses, regardless of whether it is a pathogenic agent.

As used herein, the term “Staphylococcus” means any species or strain of bacteria which is members of the genus Staphylococcus regardless of whether they are known pathogenic agents.

As used herein, “bacteremia” means the presence of viable bacteria in the blood or organs of an individual (human or other animal). “Bacteremia caused by S. aureus” or “S. aureus bacteremia” refers to bacteremia in which at least some of the bacteria in the blood or organs are S. aureus. Other species of bacteria also may be present.

Herein, “mammal” means human, bovine, goat, rabbit, mouse, rat, hamster, and guinea pig; preferred is human, rabbit, rat, hamster, or mouse and particularly preferred is human, rat, hamster, or mouse.

The term “mammals other than humans” and “non-human mammals” used herein, are synomic to each other, meaning all mammals other than humans defined above.

The terms “PILRα or PILRβ”, “Paired—immunoglobulin type 2-like receptor α or β”, “FDF03 inhibitory receptor and FDF03 activating receptor” are well known in the art. “PILR” will be used to represent “PILRα and PILRβ” unless otherwise specified. The human and mouse PILRα and PILRβ nucleotide and polypeptide sequences are disclosed in WO 1998/024906 and WO 2000/040721, respectively. The nucleic acid and amino acid sequences for human PILRα are also provided at SEQ ID NOs: 1 and 2, respectively. The nucleic acid and amino acid sequences for human PILRβ are provided at SEQ ID NOs: 3 and 4, respectively. Unless otherwise indicated or clear from the context, antibodies to PILRα and PILRβ, such as antibodies used in the experiments reported herein, are agonist antibodies, rather than antagonist antibodies.

“Antagonists of PILRβ activity” as used herein, applies to antibodies, antibody fragments, soluble domains of PILRβ, PILRβ fusion proteins, etc., that can inhibit the biological results of PILRβ activation. Fusion proteins are usually the soluble domain polypeptide of PILRβ associated with a heterologous protein or synthetic molecule, e.g., the Ig domain of an immunoglobulin.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts PILR, e.g., in the fluid phase or colloidal phase, but also situations where the agonist or antagonist does not contact the cell or the receptor.

As used herein, the term “antibody,” when used in a general sense, refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, the terms “PILR binding fragment,” “binding fragment thereof” or “antigen binding fragment thereof” encompass a fragment or a derivative of an antibody that still substantially retains its biological activity of either stimulating PILRα activity or inhibiting PILRβ activity, such inhibition being referred to herein as “PILR modulating activity.” The term “antibody fragment” or PILR binding fragment refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its PILR modulatory activity. Preferably, a binding fragment or derivative retains at least 25%, 50%, 60%, 70%, 80%, 90%, 95%, 99% or 100% (or more) of its PILR activity, although any binding fragment with sufficient affinity to exert the desired biological effect will be useful. It is also intended that a PILR binding fragment can include variants having conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in the a targeted cell.

The antibodies of the present invention also include antibodies conjugated to cytotoxic payloads, such as cytotoxic agents or radionuclides. Such antibody conjugates may be used in immunotherapy to selectively target and kill cells expressing PILR on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See, e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

As used herein, the term “hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding composition with a target where the association results in reduction in the normal Brownian motion of the binding composition, in cases where the binding composition can be dissolved or suspended in solution. “Binding composition” refers to a molecule, e.g. a binding compound, in combination with a stabilizer, excipient, salt, buffer, solvent, or additive, capable of binding to a target.

“Conservatively modified variants” or “conservative substitution” refers to substitutions of amino acids are known to those of skill in this art and may often be made even in essential regions of the polypeptide without altering the biological activity of the resulting molecule. Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Those of skill in this art recognize that, in general, single amino acid substitutions in non-essential regions of a polypeptide may not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects. See, e.g., U.S. Pat. No. 5,888,530. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

“Infection” as used herein is an invasion and multiplication of microorganisms in tissues of a subject's body. The infection or “infectious disease” may be clinically inapparent or result in local cellular injury due to competitive metabolism, toxins, intracellular replication, or antigen-antibody response. The infection may remain localized, subclinical and temporary if the body's defensive mechanisms are effective. A local invention may persist and spread by extension to become an acute, subacute, or chronic clinical infection or disease state. A local infection may also become systemic when the microorganisms gain access to the lymphatic or vascular system. Infectious diseases include bacterial, viral, parasitic, opportunistic, or fungal infections.

As used herein “antibiotic” refers to an aminoglycoside such as gentamycin or a beta-lactam such as penicillin, cephalosporin and the like. Also included are known anti-fungals and anti-virals. Antiboitics can be used with the PILR antibodies of the present invention to provide additional efficacy to clear the infection and/or prevent the development of sepsis.

As used herein, the term “isolated nucleic acid molecule” refers to a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences involved in the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

As used herein, the term “germline sequence” refers to a sequence of unrearranged immunoglobulin DNA sequences, including rodent (e.g. mouse) and human germline sequences. Any suitable source of unrearranged immunoglobulin DNA may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

To examine the extent of modulation of PILR activity, for example, samples or assays comprising a given, e.g., protein, gene, cell, or organism, are treated with a potential activating or inhibiting agent and are compared to control samples without the agent. Control samples, i.e., not treated with agent, are assigned a relative activity value of 100% Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; L1 (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence (in this case PILR) if it binds to polypeptides comprising the sequence of PILR but does not bind to proteins lacking the sequence of PILR. For example, an antibody that specifically binds to a polypeptide comprising PILR may bind to a FLAG®-tagged form of PILR but will not bind to other FLAG®-tagged proteins.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### II. General

The present invention provides methods of modulating host defense with agonists or antagonists of PILRα and PILRβ, in particular, treatment of S. aureus pulmonary infection. The results below demonstrate that upon pulmonary staphylococcal infection, activation of PILRα with an agonistic monoclonal antibody as well as deletion of PILRβ resulted in significantly improved survival and efficient clearance of the pathogen. In further support of these data, these mice also display reduced serum levels of IL-1β, TNFα and IL-6, but significantly elevated levels of IFN-γ and IL-10. In contrast, the anti-PILRβ treated and WT mice were found to be highly susceptible to S. aureus infection, displaying an intense proinflammatory response with highly elevated levels of IL-1β, TNFα and IL-6 and were thus unable to suitably control the bacterial burden in the lungs. Interestingly, mice that displayed reduced bacteremia also displayed increased neutrophil and macrophage influx at 24 h and 48 h post infection. Additionally the BAL fluid from these mice had higher amounts of KC, MIP-2, MIP-1α, which further supports the increased neutrophil and macrophage migration. The data support the view that downregulation of PILRβ results in the control of acute S. aureus-mediated lung infection by attenuating the systemic inflammatory response, thus making it an important therapeutic target for disease.

A. Deletion of the PILRβ Gene does not Alter Phenotypic Attributes in PILRβ−/− Mice

Having established a homozygous C57BL/6-PILRβ−/− strain (see below), a determination was made to ascertain if the deletion of the PILRβ gene resulted in any critical phenotypic alterations compared to WT C57BL/6 mice. Extensive analyses were performed in both female and male knockout mice with age and sex matched control WT animals in each experiment. RT-PCR analysis in various organs displayed a complete silencing of the PILRβ gene, while the expression levels of the inhibitory PILRα remained largely unaltered (see Table 2).

TABLE 2
Expression of PILRα and PILRβ in wildtype and PILRβ
knockout mice. Values are normalized to ubiquitin.

| | |
|---|---|
| PILRα | PILRβ |

| | WT | PILRβ−/− | WT | PILRβ−/− |
|---|---|---|---|---|
| Kidney | 9.74 | 3.06 | 3.94 | 0.04 |
| Liver | 130.0 | 148.0 | 135.0 | 0.05 |
| Spleen | 219.0 | 224.0 | 344.0 | 0.12 |
| Lung | 29.0 | 34.0 | 26.0 | 0.05 |
| Heart | 24.0 | 14.0 | 20.0 | 0.12 |

Furthermore, the mRNA expression levels of DAP12, TLRs and several other genes of proinflammatory cytokines and chemokines associated with acute bacterial infections remained unaffected as a result of the deletion of the PILRβ gene.

Cell surface expression of PILRα and β in leukocytes isolated from WT and knockout mouse peripheral blood using anti-mPILRα and β monoclonal antibodies was also evaluated. Cell surface staining with anti-PILRα and β antibodies displayed a 2-3 log shift in fluorescence intensity of the inhibitory PILRα and activating PILRβ respectively (compared to the isotype control) in the WT mouse cells. However, cell surface staining was observed only for PILRα in the cells from PILRβ−/− mice.

Additionally a complete blood count analysis revealed no major differences in WBC populations between the WT and PILRβ−/− mice. The hematopoietic compartment in the knockout mice and compared bone marrow and spleen isolated from PILRβ−/− and WT control littermates for expression of cell lineage markers by flow cytometry was also analyzed. No significant differences in the B cells, lymphocytes or the myeloid cell lineage were observed. These results suggest that the deletion of the mPILRβ gene does not result in any adverse phenotypic alterations in the hematopoietic compartment of these mice and also did not influence the expression levels of its inhibitory counterpart (i.e. PILRα) either at the mRNA or protein level (see Tables 3 and 4).

TABLE 3
Complete blood counts of PILRβ knockout and wild type mice

| | WT | PILRβ−/− |
|---|---|---|
| WBC (10³/μl) | 7.6 ± 2.5 | 8.7 ± 1.0 |
| Lymphocyte (10⁶/μl) | 6.2 ± 1.9 | 7.4 ± 0.90 |
| Monocyte (10⁶/μl) | 0.31 ± 0.1 | 0.26 ± 0.04 |
| Eosinophil (10⁶/μl) | 0.2 ± 0.08 | 0.21 ± 0.04 |
| Basophil (10⁶/μl) | 0.05 ± 0.02 | 0.05 ± 0.001 |
| Neutrophil (10⁶/μl) | 0.90 ± 0.35 | 0.7 ± 0.1 |

TABLE 4
Cell counts of harvested splenocytes

| | WT | PILRβ−/− |
|---|---|---|
| Splenocytes (10⁶/μl) | 89.65 | 92.0 |
| B cells (% splenocytes) | 55.71 | 57.64 |
| Dendritic cells (% splenocytes) | 3.02 | 3.08 |
| Plasmacytoid DC (% splenocytes) | 2.59 | 2.03 |
| CD4+ T cells (% splenocytes) | 44.0 | 42.9 |
| CD8+ T cells (% splenocytes) | 33.05 | 31.0 |
| NK cells (% splenocytes) | 2.7 | 2.5 |
| Neutrophils (% splenocytes) | 2.3 | 2.5 |

D. Enhanced Gene Expression of PILRα and β During S. Aureus Mediated Pneumonia

The tissue distribution of PILRα and β across various organs in naïve mice was analyzed by real-time quantitative PCR. Expression of PILRα and PILRβ were relatively high in liver and spleen, and lower in the lung, heart and kidney. Previous reports have shown a similar tissue distribution for the two receptors and have also identified PILRα and PILRβ transcripts in granulocytes, BM-DCs and macrophages, as well as PILRβ expression in NK cells (see, e.g., Shiratori, et al. supra). Because of the restriction of PILR α and β largely to cells of the innate immune system, their role in an acute bacterial infection was evaluated. The data showed that expression levels of PILRα and β were highly upregulated in lungs of mice infected with S. aureus compared to the lungs of control animals, consistent with a predominant role of PILRα and β-bearing cells in the acute response to S. aureus and raising the possibility of a role for these receptors in that response (see Table 5).

TABLE 5
Transcription levels of PILRα and PILRβ in
naïve and infected wildtype and knockout mice.

| | PILRα | PILRβ |
|---|---|---|
| infected PILRβ−/− | 576.1 ± 319 | 1.3 ± 2.1 |
| infected WT | 770.1 ± 568 | 470.9 ± 312 |
| control lung | 122.7 ± 98.0 | 80.7 ± 51 |

C. Triggering PILRβ Results in Increased Bacterial Burden, Mortality and a Damaging Inflammatory Cytokine Response

To assess the direct involvement of PILRα and PILRβ in response to bacterial infections, specific PILRα and PILRβ agonist monoclonal antibodies were used. A recently developed model of S. aureus—induced pneumonia in adult immunocompetent C57BL/6J mice that closely mimics the clinicopathological features of human disease was employed. Mice that were injected s.c. with anti-PILRβ 24 h prior to an intranasal S. aureus infection (1×10⁸ CFU/25 μl) displayed a significant increase in bacterial burden at 48 h post infection compared to mice that were injected either with anti-PILRα or isotype control. Treatment with PIRLα agonist antibodies 24 and 6 hours prior to infection showed decreased bacterial burden (see Tables 6 and 7).

TABLE 6
Bacterial burden in lungs following prophylactic antibody
treatment 24 hours prior to infection. Log₁₀ values

| | DX266 anti-mPILRβ | DX276 anti-mPILRα |
|---|---|---|
| Isotype | agonist | agonist |
| 37500 | 60000 | 3000 |
| 22500 | 45000 | 3000 |
| 30000 | 1.50E+07 | 10500 |
| 60000 | 2880000 | 900 |
| 15000 | 6000000 | 210 |
| 210000 | 390000 | 1800 |
| 33000 | 60000 | |

* p < 0.01 between Isotype and DX266 grp.
** p < 0.001 between Isotype and DX276

TABLE 7
Bacterial burden following prophylactic treatment of
FDF03 antibodies 6 h prior to S. aureus infection.

| | |
|---|---|
| Isotype | DX266 anti-mPILRβ |

| 24 h | 48 h | 24 h | 48 h |
|---|---|---|---|
| 45000 | 750000 | 67200 | 1.46E+08 |
| 6000000 | 300 | 9.00E+08 | 12000 |
| 3000 | 900 | 19200 | 48000 |
| 105000 | 60000 | 10100 | 300 |
| 1.20E+07 | 900000 | 1500 | |
| 120000 | 18000 | 1200 | |
| | | 9.00E+08 | |

In order to obtain a better understanding of the roles of PILRα and PILRβ in acute bacterial infections, a therapeutic approach was used in which the agonist antibodies were administered i.v. 2 h after intranasal infection. Again, mice injected with anti-PILRβ mAb (agonist antibody) were more susceptible to bacterial infection, while those treated with anti-PILRα agonist mAb were able to clear the infection better within 48 h compared to the control mice (see Table 8). The anti-PILRβ treated mice displayed significantly higher bacteraemia (p≦0.036) with a 75% mortality rate 48 h post infection. In contrast the anti-PILRα treated group displayed significantly reduced staphylococci in the lungs (p≦0.031), with no apparent difference in their survival compared to the control mice.

TABLE 8
Bacterial burden in lungs following therapeutic treatment
with antibodies 2 hours post infection (Log₁₀ values)

| Isotype | anti-PILRβ | anti-PILRα |
|---|---|---|
| 1200 | 84000 | 1350 |
| 3750 | 2.10E+08 | 36000 |
| 4500 | 2304000 | 690 |
| 22500 | 300000 | 60 |
| 3600 | 6.00E+07 | 60 |
| 7500 | 90000 | 990 |
| 601500 | | 840 |
| 9600 | | 600 |
| 60000 | | 600 |
| 30000 | | 2100 |
| 2202000 | | 300 |
| 90000 | | 150 |
| | | 3000 |
| | | 3300 |
| Avg = 253012 | Avg = 45463000 | Avg = 3574 |

Effective clearance of bacterial infection in the lungs requires a vigorous and appropriate recruitment of neutrophils. In order to determine neutrophil recruitment, total myeloperoxidase (“MPO”) activity was measured. Although the MPO levels were similar among the groups at 6 h, at 24 h post infection lung tissues from mice treated with anti-PILRβ had significantly reduced levels of MPO, while in anti-PILRα treated mice the MPO levels were considerably higher compared to the control mice. Additionally, at both 24 and 48 h post inoclulation, increased systemic levels of proinflammatory cytokines such as TNFα, IL-1β, IL-6 in anti-PILRβ treated mice corresponded with their increased susceptibility to S. aureus infection. In contrast, anti-PILRα treated mice displayed reduced levels of proinflammatory mediators and significantly increased amounts of cytokines such as IL-10, IL-12p70 and INFγ, cytokines that promote phagocytic uptake and killing of S. aureus. An increase in IL-15 in these mice was also observed, suggesting a role for NK cells and macrophages in clearing the bacteria (Gonzalez-Juarrero, et al. (2003) J. Immunol. 171:3128-3135).

TABLE 9
MPO concentrations 6 hours post infecton

| | uninfected | Isotype ctrl | anti-PILRβ | anti-PILRα |
|---|---|---|---|---|
| | 7.7 | 78.58 | 123.83 | 81.45 |
| | 8.6 | 130.76 | 99.10 | 84.08 |
| | | 151.25 | 102.79 | 95.02 |
| | | 98.14 | 71.80 | 90.90 |
| Avg and SD | 8.2 ± 0.7 | 114.68 ± 32.0 | 99.38 ± 21.1 | 87.86 ± 6.2 |

TABLE 10
MPO concentratons 24 hours post infection

| | uninfected | Isotype ctrl | anti-PILRβ | anti-PILRα |
|---|---|---|---|---|
| | 7.7 | 86.14 | 38.05 | 151.85 |
| | 8.9 | 46.28 | 30.73 | 93.51 |
| | | 53.87 | 21.80 | 65.66 |
| | | 74.32 | | 89.36 |
| Avg and SD | 8.3 ± 0.8 | 65.15 ± 18.1 | 30.12 ± 8.1 | 100.09 ± 36.6 |

E. Downregulation of PILRβ Protects Against Acute S. Aureus Lung Infection

To evaluate the in vivo role of PILRβ during S. aureus infection, WT and PILRβ−/− mice were infected intranasally with 1×10⁸ CFU/25 μl/mouse. The severity of the infection was monitored by assessing both survival rates and bacterial accumulation in the lungs. PILRβ−/− mice were more resistant to S. aureus infection than the WT mice, with an improved rate of bacterial clearance (p≦0.05 and 0.04) and reduced mortality (p≦0.023).

TABLE 11
Bacterial burden in wild-type and PILRβ knockout mice

| WT | PILRβ−/− | WT | PILRβ−/− | WT | PILRβ−/− |
|---|---|---|---|---|---|
| 6 h | | 24 h | | 48 h | |
| 4.36E+08 | 2181818 | 3000 | 300 | 120 | 30 |
| 2135231 | 2135231 | 4800 | 1020 | 6.90E+03 | 30 |
| 7.25E+07 | 36253.78 | 6000 | 218.1818 | 210 | 90 |
| 2500000 | 3750000 | 5.08E+07 | 960.8541 | 2.55E+05 | 2820 |
| 874635.6 | 612244.9 | 5952381 | 29365.56 | 285000 | 360 |
| 9000000 | 4110000 | 6641222 | 2500 | 6.00E+07 | 120 |
| 1.59E+07 | 1.47E+07 | 212766 | 874.6356 | 6.00E+06 | 27300 |
| 4590000 | 1290000 | 131579 | 1686.747 | 321000 | 8430 |
| 1.41E+07 | 1500000 | 4.35E+08 | 1098.901 | 4.44E+04 | 12900 |
| 7800000 | | 1.14E+07 | 4979.25 | 27000 | 1830 |
| | | 1.76E+07 | 254237 | 75000 | 1980 |
| | | 23076.92 | 212500 | 1.36E+07 | 1200 |
| | | 193548.4 | 524017 | 100200 | 72 |
| | | 12765.96 | 125769 | 12900 | 72 |
| | | 5400000 | 212500 | 300 | 1950 |
| | | 1.80E+08 | | 300 | 6000 |
| | | 1.80E+08 | | 1880.878 | 1800 |
| | | | | 31067.96 | 9000 |
| | | | | 8928.571 | 3600 |
| | | | | 2828.572 | 2400 |
| | | | | 570.6522 | 22655.6 |
| | | | | | 1525.42 |
| | | | | | 875 |
| | | | | | 262.009 |
| | | | | | 1384.62 |

In addition, systemic levels of IL-6, IL-1β, TNFα and MCP-1 were compared between PILRβ−/− and control mice at 6, 24 and 48 h post infection. S. aureus infection in PILRβ−/− mice resulted in remarkably reduced levels of these proinflammatory cytokines compared to WT mice. Notably, in PILRβ−/− mice a significantly decreasing trend was observed in serum levels of IL-1β (p≦0.037), IL-6 (p≦0.04), MCP-1 (p≦0.034) and TNFα at 6, 24 and 48 h post-infection. Consistent with the reduced bacterial load and improved survival, serum samples from these knockout mice showed pronounced levels of immune mediators such as IFNγ, IL-12p70. It was also interesting to note that the PILRβ−/− mice displayed sustained and elevated levels of both IFNγ and IL-12p70, suggesting a critical role for these cytokines in mobilizing and clearing the infection. While IL-15 concentrations were found to be increased in the knockout mice, the level was not significantly different to WT animals. Interestingly however, serum levels of KC (GROα) were found to be elevated in both groups at 6 h followed by a significant decrease by 24 and 48 h. Also, serum levels of MIP-2 in WT mice were significantly higher than in knockout mice at 6 h post infection. At 24 and 48 h post infection MIP-2 serum concentrations were considerably reduced in both groups. Furthermore, expression of IL-1β transcripts by real-time quantitative PCR from WT infected lungs were considerably higher than observed for PILRβ−/− mice. In contrast, levels of IFN-γ and IL-12p40 transcripts are elevated in the lungs of knockout mice 48 h post infection.

F. Effect of PILRβ Deficiency on Cytokine and Chemokine Production in BAL Fluids

Levels of proinflammatory chemokines KC, MIP-2, MIP-1α, and RANTES in the BAL fluid of infected mice were measured. Levels were elevated at 6 and 24 h postinoculation with S. aureus in the PILRβ−/− mice compared to the WT control animals. Notably, in the WT mice an increasing trend was observed in the levels of TNFα and IL-1β both at 6 and 24 h post inoclulation and only at 6 h for IL-6 and MCP-1. To counteract the damaging effect of these proinflammatory cytokines, significantly higher levels of IFN-γ and IL-10 (p≦0.0008 and 0.035 respectively) were observed in the BAL samples of the knockout mice at 24 h post infection. However the levels of these cytokines were found to be noticeably lower in the BAL fluid of the WT mice throughout the observation period.

G. Histopathological Findings

To assess the consequences of S. aureus infection in the airways of lungs of infected mice, lung specimens were harvested at 6, 24 and 48 h post infection from WT and PILRβ−/− mice, stained with H&E and examined microscopically. Lungs from 6 h-infected mice (FIG. 7 6 h) had some neutrophilic infiltration with various degrees of severity in both groups. However, at 24 h and 48 h post infection, more frequent confluent foci of cellular infiltration were observed in the lungs of PILRβ−/− mice compared to WT mice, suggesting suitable recruitment of PMNs to the site of infection for effective clearance of bacteria.

H. Effective and Early Recruitment of Neutrophils into the Bronchoalveolar Space Protects PILRβ−/− Mice Against S. Aureus Pneumonia

Neutrophil sequestration is an essential component of antibacterial defense during an innate immune response. To understand the cause for the profound protective phenotype observed in the PILRβ−/− mice during an intransal S. aureus infection, flow cytometry was used to define the influx of cells into the lungs during the acute phase of pulmonary S. aureus infection. The phenotype and composition of cells in the lungs were monitored in naive WT and PILRβ−/− mice as well as in infected mice 24 and 48 h post infection. Cells in the lungs of naïve and challenged mice were initially analyzed according to their FSC and SSC characteristics. Cells that clustered as FCS^{low}/SSC^{low} were gated as R2 and those that came together as FCS^{high}/SSC^{hi} were gated as R3. Under naïve conditions the R2 population was almost twice the size of the R3 population both in the WT and PILRβ−/− mice and was composed mainly of lymphocytes and monocytes. However, after mice received the bacterial challenge, the percentages of cells in the R2 and R3 gates were completely reversed.

Further analysis of cells in the R2 gate showed that cells were mainly CD3⁺, CD11b⁺/Gr-1^{lo-int} (small macrophages), CD11b⁺/CD11c^{lo} (monocytes and small macrophages and CD11b⁺/F/480^{int} (alveolar macrophages) for the naïve mice. Analysis of the R3 gate in naïve mice was primarily dominated by the resident alveolar macrophages defined as CD11b⁺/F/480^{int} and CD11b⁻/CD11c⁺ (35%) and a lower percentage of CD11b⁺/Gr1⁺ (18%). Previous reports have also characterized the CD11b⁺/F/480^{int} and CD11b⁻/CD11c⁺ as small macrophages and alveolar macrophages, respectively (see, e.g., Gonzalez-Juarrero, et al. (2003) J. Immunol. 171:3128-3135). However, as a result of infection, a significant increase was observed in CD11b⁺/GR-1^{lo-int} and CD11b⁺/GR-1^{hi} both in the R2 and R3 gates at 24 h. Both these populations were significantly higher among the PILRβ−/− mice compared to the WT mice. Furthermore, a sustained increase in the number of CD11b⁺/GR-1^{lo-int} cells (macrophages) in PILRβ−/− mice was observed even 48 h post infection in the R3 cell populations. Another striking observation was a predominant increase of CD11b⁺/GR-1^{hi} in R2 and R3 gates and a significant decrease in the CD11b⁻/CD11c⁺ and CD11b⁺/F/480^{int} cells in the knockout mice. This suggests that upon infection the PILRβ−/− mice were able to initiate and maintain an appropriate influx of neutrophils and macrophages for the effective clearance of the S. aureus bacteria in the lung, thus adding further credence to the importance of neutrophils in combating acute pulmonary bacterial infection. In addition cells were also stained with anti-CD3, anti-CD8, anti-CD4 and anti-NK1.1 mAbs, but no apparent difference in cell numbers were observed between the two groups of mice under naive and infected conditions.

More than 20 receptors pairs consisting of highly related activating and inhibitory isoforms have been identified so far, suggesting that the pairing of activation and inhibition is critical to the amplification and termination of an immune response (see, e.g., Lanier, et al. (2001) supra; and Torii, et al. (2008) J. Immunol. 181:4229-4239). PILRα and PILRβ are a pair of novel immune regulatory receptors with opposing signaling capabilities and are expressed primarily on neutrophils, macrophages and dendritic cells (see, e.g., Fournier, et al. (2000) supra). However, very little is known regarding the regulation of their expression and their involvement in host responses to S. aureus infection. The above data demonstrate a direct involvement for both PILRα and PILRβ in tightly regulating the innate immune response during pulmonary S. aureus infection.

During a bacterial infection, execution of a successful innate immune response begins with the recognition of invading bacteria by highly conserved pattern recognition receptors—the TLRs present on the surface of the myeloid cells (see, e.g., Takeda, et al. (2003) Annu. Rev. Immunol. 21:335-376). In addition to the TLRs, it has also been reported that many additional innate immune receptors also participate in fine-tuning the regulatory mechanism. Recently it was shown that paired Ig-like receptors comprising the activating PIR-A and inhibitory PIR-B are able to recognize the S. aureus pathogen and regulate TLR-mediated cytokine production (see, e.g., Nakayama, et al. (2007) J. Immunol. 178:4250-4259. Additionally, genetic deletion of PIR-B significantly impaired recognition of S. aureus and enhanced TLR-mediated inflammatory responses in PIR-B−/− BM-derived macrophages.

Like MDL-1 and TREM-1 (see, e.g., Bakker, et al. (1999) Proc. Natl. Acad. Sci. 96:9792-9796; and Bouchon, et al. (2000) J. Immunol. 164:4991-4995), PILRβ associates with DAP12 to transmit an activation signal (see, e.g., Shiratori, et al. supra). In contrast, upon activation or ligand interaction PILRα transduces an inhibitory signal through the phosphorylation of its ITIM motifs. Initial studies have implicated the same ligand for both of these receptors in mice (see, e.g., Shiratori, et al. supra). Thus the overall expression of the receptors as well as that of the ligand determines an immune activation or suppression mechanism. While too small a response makes the host susceptible to infection, too great a response may result in lethal systemic inflammation. As noted above, agonist anti-PILRα and anti-PILRβ mAbs as well as a PILRβ−/− mouse were used to assess the involvement of PILRα and PILRβ in S. aureus-mediated lung infection. The results show that independent triggering of these two receptors can induce opposite immune responses during an S. aureus infection. While anti-PILRα treated mice were better able to clear the infection, anti-PILRβ treated animals were highly susceptible to the pathogen and displayed an increased bacterial burden accompanied by a higher mortality rate. Furthermore, increased bacteremia and mortality in these mice was also associated with a profound inflammatory response with increased levels of proinflammatory cytokines such as IL-1β, IL-6 and TNFα and significantly reduced amounts of cytokines such as IFN-γ, IL-12p70, IL-10 and IL-15 as detected in the serum of these mice. The levels of these cytokines were completely reversed in the anti-PILRα treated group of mice.

In accordance with these findings, the PILRβ−/− mice were also found to be more resistant to S. aureus compared to WT mice and consequently exhibited decreased bacterial burdens and greater survival. This striking phenotype was associated with remarkably reduced levels of different proinflammatory cytokines, in particular IL-1β, which is considered the hallmark of acute lung injury (see, e.g., Bubeck-Wardenburg and Scheewind (2008) J. Exp. Med. 205:287-294). Similar observations were made with respect to TREM-1, wherein blocking TREM-1 using an LP17 peptide appeared to be beneficial during P. aeruginosa pneumonia in rats (see, e.g., Gibot et al. (2006) J. Infect. Dis. 194:975-981).

Taken together, the results from antibody treatment and PILRβ−/− mice suggest that overactivation of DAP12/PILRβ pathway can have a deleterious consequence, resulting in uncontrolled inflammation leading to septic shock, organ failure and ultimately death. Interestingly, Akoi et al (2004) Infect. Immun. 72:2477-2483) have demonstrated that type 1 cytokines such as TNFα and IFN-γ are closely associated with the kinetics of expression of DAP12 and some of its associating molecules. In particular it was observed that TNFα was required for mycobacterially induced MDL-1 expression, while IFN-γ suppressed expression of MDL-1 and TREM-1 during mycobacterial infection. Furthermore, the above data also indirectly demonstrate that increased levels of TNFα and IL1-β in the serum of anti-PILRβ-treated or WT mice could play a role in governing the overall increased expression of PILRβ, resulting in increased inflammation, while the elevated levels of IFN-γ may be responsible for increased S. aureus induced expression of PILRα, which may aid towards controlling and suppressing the acute inflammatory response. Thus these findings confirm previous observations (see, e.g., Aoki et al. (2004) supra; and Aoki and Xing (2004) Expert Opin. Emerg. Drugs 9:223-236) suggest that these cytokines may be involved in the regulation of expression of DAP12 and its associating molecules. Moreover, since DAP12 is constitutively expressed at high levels in the lung, the relative expression and availability of PILRβ like other DAP12 associating receptors may be responsible for controlling the DAP12 signaling pathway during S. aureus infection in the lung.

Even as different immunoreceptors deployed by the innate immune system are able to regulate and target inflammatory responses by virtue of their relative expression, neutrophils and macrophages harboring these receptors form key mediators of innate immunity by providing a first line of host defense. In the lung, the primary defense mechanism is mediated through a local inflammatory response to external pathogens by neutrophils, monocytes and macrophages (see, e.g., Nizet (2007) J. Allergy and Clin. Immunol. 120:13-22; and Richeldi et al. (2004) Eur. Respir. 24:247-250). As noted above there was a significant increase in the number of neutrophils both in the lungs of the PILRβ−/− and anti-PILRα-treated mice. An increase in the MPO levels at 24 h in the anti-PILRα treated mice as well as a significant increase in the CD11b⁺/GR-1^{hi} population of cells in the knockout mice are clear indications for the increase in neutrophil numbers.

A correlation between this increased neutrophilic infiltration and elevated levels of chemokines such as MIP-2, KC, MIP-1α and RANTES was observed in the BAL fluids of the PILRβ−/− mice. The neutrophilic population peaked at 24 h and returned to normal levels by 48 h. Interestingly, the macrophage population defined by CD11b⁺/Gr1^{lo} continued to remain significantly higher in the infected knockout mice even at 48 h, suggesting that after 48 h of infection the macrophages play a critical role in clearing bacteria and apoptosized neutrophils. In support of this observation the levels of IFN-γ in these mice were also notably elevated both in the serum and BAL fluids further reinstating the association of INF-γ with increased phagocytic uptake and killing of S. aureus by neutrophils and macrophages (see, e.g., Zhao, et al. (1998) Immunology 93:80-85). Conversely, much reduced MPO levels and neutrophil infiltration was observed among the anti-PILRβ treated and WT infected mice respectively. Interestingly, 6 h post infection levels of MPO were found to be similar among different antibody treated animals, but the levels were found to dramatically decline at 24 h in the anti-PILRβ treated animals. This could suggest that although the initial recruitment of neutrophils to the site of infection is similar, an overactivation in the DAP12/PILRβ pathway and resultant inflammatory environment prevents further effective recruitment of neutrophils. In this regard, the observation above is in agreement with previous studies demonstrating an impairment in neutrophil migration during severe infection coupled with increased bacteremia and mortality (see, e.g., Alves-Filho, et al. (2008) Shock 30 Suppl 1:3-9). Furthermore, this striking correlation in impaired accumulation of bronchoalveolar neutrophils between the anti-PILRβ treated animals and the WT animals could imply a reduction in the emigration of neutrophils from the blood along with a defective migration across the lung epithelium resulting in increased bacterial burden and mortality in these mice.

Based on the above data, it is believed that upon S. aureus insult, an initial neutrophil migration occurs at the site of infection. In the WT group or in mice treated with anti-PILRβ agonistic mAb, the bacterial components cause an upregulation of TLRs and different immune regulatory receptors and their unknown endogenous ligands present on these neutrophils. Overexpression and the synergistic effect between the TLRs and the DAP12-associating activating receptors such as PILRβ results in activation of NFκ-B signaling pathway resulting in highly increased levels of proinflammatory cytokines such as IL-1β, TNFα and IL-6. This exacerbated proinflammatory response impairs further neutrophil or macrophage recruitment resulting in reduced anti-bacterial activity, increased tissue damage and susceptibility to infection. Although the mechanism of activation versus inhibition is not completely understood, it can be speculated that, deletion of the activating PILRβ in the knock out mice or triggering of the PILRα receptor with the anti-PILRα agonistic antibody, skews the balance of the immune response to a more suppressive state. This in turn results in reduced levels of proinflammatory cytokines (IL-1β, TNFα, and IL-6) but elevated levels of other cytokines and chemokines that allows for suitable and continued neutrophil and macrophage recruitment, effective clearance of bacteria and ultimately improved survival. In support of the above hypothesis, we observed increased levels of chemokines such KC, MIP-2, MCP-1 and RANTES in the BAL fluid as well as increased levels of anti-inflammatory mediators such as IFN-γ, IL-12 and IL-10 in the serum and BAL fluid of PILRβ−/− mice both at 24 and 48 h post infection. Previous studies have also demonstrated an important role for IL-12 in neutrophil recruitment in response to L. pneumophilia infection (see, e.g., Tateda et al (2001) Infect. Immun. 69:2017-2024).

Modulation of the PILRα and β pathways by either triggering PILRα or downregulating PILRβ attenuates the local inflammatory response during S. aureus-mediated lung pneumonia. These results are be indicative of the therapeutic value of PILRα agonists or PILRβ antagonists to treat other S. aureus infection throughout the body, including the skin. In particular the agonists/antagonists can be used to treat drug-resistant forms of the microbial infection.

### III. Generation of PILR Antibodies

Any suitable method for generating monoclonal antibodies may be used. For example, a recipient may be immunized with PILR or a fragment thereof. Any suitable method of immunization can be used. Such methods can include adjuvants, other immunostimulants, repeated booster immunizations, and the use of one or more immunization routes. Any suitable source of PILR can be used as the immunogen for the generation of the non-human antibody of the compositions and methods disclosed herein. Such forms include, but are not limited whole protein, peptide(s), and epitopes generated through recombinant, synthetic, chemical or enzymatic degradation means known in the art. In preferred embodiments the immunogen comprises the extracellular portion of PILR.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Any suitable method can be used to elicit an antibody with the desired biologic properties to modulate PILR signaling. It is desirable to prepare monoclonal antibodies (mAbs) from various mammalian hosts, such as mice, rats, other rodents, humans, other primates, etc. Description of techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a antigen binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. supra; and Ward et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance that provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989)Proc. Nat'l Acad. Sci. USA 86:10029-10033; or made in transgenic mice, see Mendez et al. (1997) Nature Genetics 15:146-156. See also Abgenix and Medarex technologies.

Antibodies or binding compositions against predetermined fragments of PILR can be raised by immunization of animals with conjugates of the polypeptide, fragments, peptides, or epitopes with carrier proteins. Monoclonal antibodies are prepared from cells secreting the desired antibody. These antibodies can be screened for binding to normal or defective PILR. These monoclonal antibodies will usually bind with at least a K_{d} of about 1 μM, more usually at least about 300 nM, 30 nM, 10 nM, 3 nM, 1 nM, 300 pM, 100 pM, 30 pM or better, usually determined by ELISA.

Any suitable non-human antibody can be used as a source for the hypervariable region. Sources for non-human antibodies include, but are not limited to, murine (e.g. Mus musculus), rat (e.g. Rattus norvegicus), Lagomorphs (including rabbits), bovine, and primates. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance of the desired biological activity. For further details, see Jones et al. (1986) Nature 321:522-525; Reichmann et al. (1988) Nature 332:323-329; and Presta (1992) Curr. Op. Struct. Biol. 2:593-596.

Anti-PILR antibodies of the present invention may be screened to ensure that they are specific for only one of PILRα and PILRβ as follows. Clearly, anti-PILRα antibodies are raised using immunogen comprising PILRα, or an immunogenic fragment thereof, and anti-PILRβ antibodies are raised using immunogen comprising PILRβ, or an immunogenic fragment thereof. To confirm that the resulting anti-PILR antibodies do not cross-react with the other form of PILR, a competition ELISA may be used. Briefly, the immunogen used to raise the antibody is bound to a well on a plate. Candidate antibodies are added to the wells either alone, or in the presence of varying concentrations of PILRα and PILRβ or fragments thereof. The ratio of PILRα to PILRβ necessary to achieve a given level of inhibition of binding (e.g. 50% reduction) reflects the PILRα-specificity of the candidate antibody. In the case of antibodies raised against PILRβ, or an antigenic fragment thereof, the ratio can more conveniently be expressed as the PILRβ-specificity (the ratio of PILRβ to PILRα). Non-cross-reactive anti-PILR antibodies may exhibit PILRα- or PILRβ-specificities of about two, five, ten, 30, 100, 300, 1000 or more.

Note that it is not necessarily essential that an anti-PILR antibody be non-cross-reactive with the other form of PILR, provided that the antibody nonetheless provides therapeutic benefit. For example, a bispecific agonist antibody against both PILRα and PILRβ may give results similar to those seen with an agonist of PILRα alone, and thus may be therapeutically beneficial. Accordingly, a PILRα agonist need not necessarily be completely non-cross-reactive with PILRβ to show beneficial effect.

Anti-PILR antibodies may also be screened to identify antagonists of PILRβ or agonists of PILRα. One screen for PILRβ antagonists is based on use of PILRβ agonists, such as the putative natural ligand CD99 (SEQ ID NOs: 6 and 8) or agonist anti-PILRβ antibodies (e.g. DX266), to induce degranulation of mast cells. See Example 18. Accordingly, antagonists of PILRβ can be identified by screening for agents (e.g. antibodies) that block this agonist-induced degranulation.

Similarly, agonists of the inhibitory PILRα receptor can be identified based on their ability to suppress mast cell degranulation, for example degranulation induced by agonists of the activating receptor PILRβ or agonists of other activating receptors, such as CD200RL1. See Example 18.

Bispecific antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

The parental and engineered forms of the antibodies of the present invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al., (1999) (Bioconj. Chem. 10:973-981) discloses PEG conjugated single-chain antibodies. Wen et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., (1962) Nature 144:945; David et al., (1974) Biochemistry 13:1014; Pain et al., (1981) J. Immunol. Meth. 40:219; and Nygren, J., (1982) Histochem. and Cytochem. 30:407. Methods for conjugating antibodies and proteins are conventional and well known in the art.

### IV. Nucleic Acid-Based Antagonists of PILRβ

An antagonist of PILRβ also includes nucleic acid-based antagonists that reduce the expression of PILRβ, such as antisense nucleic acids and siRNA. See, e.g., Arenz and Schepers (2003) Naturwissenschaften 90:345-359; Sazani and Kole (2003) J. Clin. Invest. 112:481-486; Pirollo et al. (2003) Pharmacol. Therapeutics 99:55-77; Wang et al. (2003) Antisense Nucl. Acid Drug Devel. 13:169-189. Design of such antagonists is within the skill in the art in light of the known sequence of the mRNA encoding PILRβ, which is available at NCBI Nucleic Acid Sequence Database Accession Numbers NM_{—}013440.3, and is provided herein at SEQ ID NO: 3.

Methods of producing and using siRNA are disclosed, e.g., at U.S. Pat. Nos. 6,506,559 (WO 99/32619); 6,673,611 (WO 99/054459); 7,078,196 (WO 01/75164); 7,071,311 and PCT publications WO 03/70914; WO 03/70918; WO 03/70966; WO 03/74654; WO 04/14312; WO 04/13280; WO 04/13355; WO 04/58940; WO 04/93788; WO 05/19453; WO 05/44981; WO 03/78097 (U.S. patents are listed with related PCT publications). Exemplary methods of using siRNA in gene silencing and therapeutic treatment are disclosed at PCT publications WO 02/096927 (VEGF and VEGF receptor); WO 03/70742 (telomerase); WO 03/70886 (protein tyrosine phosphatase type IVA (Pr13)); WO 03/70888 (Chk1); WO 03/70895 and WO 05/03350 (Alzheimer's disease); WO 03/70983 (protein kinase C alpha); WO 03/72590 (Map kinases); WO 03/72705 (cyclin D); WO 05/45034 (Parkinson's disease). Exemplary experiments relating to therapeutic uses of siRNA have also been disclosed at Zender et al. (2003) Proc. Nat'l. Acad. Sci. (USA) 100:7797; Paddison et al. (2002) Proc. Nat'l. Acad. Sci. (USA) 99:1443; and Sah (2006) Life Sci. 79:1773. siRNA molecules are also being used in clinical trials, e.g., of chronic myeloid leukemia (CML) (ClinicalTrials.gov Identifier: NCT00257647) and age-related macular degeneration (AMD) (ClinicalTrials.gov Identifier: NCT00363714).

Although the term “siRNA” is used herein to refer to molecules used to induce gene silencing via the RNA interference pathway (Fire et al. (1998) Nature 391:806), such siRNA molecules need not be strictly polyribonucleotides, and may instead contain one or more modifications to the nucleic acid to improve its properties as a therapeutic agent. Such agents are occasionally referred to as “siNA” for short interfering nucleic acids. Although such changes may formally move the molecule outside the definition of a “ribo” nucleotide, such molecules are nonetheless referred to as “siRNA” molecules herein. For example, some siRNA duplexes comprise two 19-25 nt (e.g. 21 nt) strands that pair to form a 17-23 basepair (e.g. 19 base pair) polyribonucleotide duplex with TT (deoxyribonucleotide) 3′ overhangs on each strand. Other variants of nucleic acids used to induce gene silencing via the RNA interference pathway include short hairpin RNAs (“shRNA”), for example as disclosed in U.S. Pat. App. Publication No. 2006/0115453.

The sequence of the opposite strand of the siRNA duplexes is simply the reverse complement of the sense strand, with the caveat that both strands have 2 nucleotide 3′ overhangs. That is, for a sense strand “n” nucleotides long, the opposite strand is the reverse complement of residues 1 to (n-2), with 2 additional nucleotides added at the 3′ end to provide an overhang. Where an siRNA sense strand includes two U residues at the 3′ end, the opposite strand also includes two U residues at the 3′ end. Where an siRNA sense strand includes two dT residues at the 3′ end, the opposite strand also includes two dT residues at the 3′ end.

The use of complimentary sequences to arrest translation of mRNAs was described in the late 1970s. See, e.g., Paterson et al. (1977) Proc. Natl. Acad. Sci. (USA) 74:4370-4374; Hastie & Held (1978) Proc. Natl. Acad. Sci. (USA) 75: 1217-1221 and Zamecnik & Stephenson (1978) Proc. Natl. Acad. Sci. (USA) 75:280-284. However, the use of antisense oligonucleotides for selective blockage of specific mRNAs is of recent origin. See, e.g., Weintraub et al. (1985) Trends Genet. 1:22-25 (1985); Loke et al. (1989) Proc. Natl. Acad. Sci. (USA) 86:3474-3478; Mulligan et al. (1993) J. Med. Chem. 36:1923-1937 (1993); and Wagner (1994) Nature 372:333-335. The mechanism of antisense inhibition in cells was previously analyzed and the decrease in mRNA levels mediated by oligonucleotides was shown to be responsible for the decreased expression of several proteins. See Walder & Walder (1988) Proc. Natl. Acad. Sci. (USA) 85:5011-5015; Dolnick (1991) Cancer Invest. 9:185-194; Crooke & LeBleu (1993) Antisense Research and Applications, CRC Press, Inc., Boca Raton, Fla.; Chiang et al. (1991) J. Biol. Chem. 266:18162-18171; and Bennett et al. (1994) J. Immunol. 152:3530-3540. The use of antisense oligonucleotides is recognized as a viable option for the treatment of diseases in animals and man. For example, see U.S. Pat. Nos. 5,098,890; 5,135,917; 5,087,617; 5,166,617; 5,166,195; 5,004,810; 5,194,428; 4,806,463; 5,286,717; 5,276,019; 5,264,423; 4,689,320; 4,999,421 and 5,242,906, which teach the use of antisense oligonucleotides in a variety of diseases including cancer, HIV, herpes simplex virus, influenza virus, HTLV-HI replication, prevention of replication of foreign nucleic acids in cells, antiviral agents specific to CMV, and treatment of latent EBV infections.

An antisense nucleic acid can be provided as an antisense oligonucleotide. See, e.g., Murayama et al. (1997) Antisense Nucleic Acid Drug Dev. 7:109-114. Genes encoding an antisense nucleic acid can also be provided; such genes can be formulated with a delivery enhancing compound and introduced into cells by methods known to those of skill in the art. For example, one can introduce a gene that encodes an antisense nucleic acid in a viral vector, such as, for example, in hepatitis B virus (see, e.g., Ji et al. (1997) J. Viral Hepat. 4:167-173); in adeno-associated virus (see e.g., Xiao et al. (1997) Brain Res. 756:76-83; or in other systems including, but not limited, to an HVJ (Sendai virus)-liposome gene delivery system (see, e.g., Kaneda et al. (1997) Ann. N.Y. Acad. Sci. 811:299-308); a “peptide vector” (see, e.g., Vidal et al., (1997) CR Acad. Sci. III 32:279-287); as a gene in an episomal or plasmid vector (see, e.g., Cooper et al. (1997) Proc. Natl. Acad. Sci. (U.S.A.) 94:6450-6455, Yew et al. (1997) Hum Gene Ther. 8:575-584); as a gene in a peptide-DNA aggregate (see, e.g., Niidome et al. (1997) J. Biol. Chem. 272:15307-15312); as “naked DNA” (see, e.g., U.S. Pat. No. 5,580,859 and U.S. Pat. No. 5,589,466); in lipidic vector systems (see, e.g., Lee et al. (1997) Crit. Rev. Ther. Drug Carrier Syst. 14:173-206); polymer coated liposomes (U.S. Pat. Nos. 5,213,804 and 5,013,556); cationic liposomes (U.S. Pat. Nos. 5,283,185; 5,578,475; 5,279,833; 5,334,761); gas filled microspheres (U.S. Pat. No. 5,542,935), ligand-targeted encapsulated macromolecules (U.S. Pat. Nos. 5,108,921; 5,521,291; 5,554,386; and 5,166,320).

### V. Pharmaceutical Compositions

To prepare pharmaceutical or sterile compositions including PILR antibodies, the polypeptide analogue or mutein, antibody thereto, or nucleic acid thereof, is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions. See, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms Parenteral Medications, Marcel Dekker, N Y; Lieberman, et al. (eds.) (1990) Pharmaceutical Dosage Forms: Tablets, Marcel Dekker, N Y; Lieberman et al. (eds.) (1990) Pharmaceutical Dosage Forms: Disperse Systems, Marcel Dekker, N Y; Weiner and Kotkoskie (2000) Excipient Toxicity and Safety, Marcel Dekker, Inc., New York, N.Y.

Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration is not particularly important. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is substantially derived from the same species as the animal targeted for treatment (e.g. a humanized antibody for treatment of human subjects), thereby minimizing any immune response to the reagent.

Antibodies, antibody fragments, and cytokines can be provided by continuous infusion, or by doses at intervals of, e.g., one day, 1-7 times per week, one week, two weeks, monthly, bimonthly, etc. Doses may be provided intravenously, subcutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

As used herein, “inhibit” or “treat” or “treatment” includes a postponement of development of the symptoms associated with a microbial infection and/or a reduction in the severity of such symptoms that will or are expected to develop. Thus, the terms denote that a beneficial result has been conferred on a vertebrate subject with an microbial infection, or with the potential to develop such a disease or symptom.

As used herein, the term “therapeutically effective amount” or “effective amount” refers to an amount of an PILR-specific binding compound, e.g. and antibody, that when administered alone or in combination with an additional therapeutic agent to a cell, tissue, or subject is effective to prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent, e.g., a cytokine, antibody, steroid, chemotherapeutic agent, antibiotic, or radiation, are well known in the art, see, e.g., Hardman et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA. Antibiotics can include known antibacterial, anti-fungal, and anti-viral agents. Antibacterial agents can include, but are not limited to beta lactam agents that inhibit of cell wall synthesis, such as penicillins, cephalosporins, cephamycins, carbopenems, monobactam; and non beta lactam agents that inhibit cell wall synthesis, such as vancomycin and teicoplanin. Other antibiotics can inhibit cellular activity such as protein and nucleic acid synthesis. These agents include, but are not limited to, macrolides, tetracyclines, aminoglycosides, chloramphenicol, sodium fusidate, sulphonamides, quinolones, and azoles.

Known anti-fungals include, but are not limited to, allylamines and other non-azole ergosterol biosynthesis inhibitors, such as terbinafine; antimetabolites, such as flucytosine; azoles, such as fluconazole, itraconazole, ketoconazole, ravuconazole, posaconazole, and voriconazole; glucan synthesis inhibitors, such as caspofungin, micafungin, and anidulafungin; polyenes, such as amphotericin B, amphotericin B Lipid Complex (ABLC), amphotericin B colloidal dispersion (ABCD), liposomal amphotericin B (L-AMB), and liposomal nystatin; and other systemic agents, such as griseofulvin.

Anti-virals include any drug that destroys viruses. Antivirals may include interferons which function to inhibits replication of the virus, protease inhibitors, and reverse transcriptase inhibitors.

Typical veterinary, experimental, or research subjects include monkeys, dogs, cats, rats, mice, rabbits, guinea pigs, horses, and humans.

### VI. Uses

The present invention provides methods for using anti-PILR antibodies and fragments thereof for the treatment and diagnosis of, e.g., infectious diseases.

The present invention provides methods for diagnosing the presence of a microbial infection or cancer by analyzing expression levels of PILR in test cells, tissue or bodily fluids compared with PILR levels in cells, tissues or bodily fluids of preferably the same type from a control. As demonstrated herein, an increase in level of PILR expression, for example, in the patient versus the control is associated with the presence of cancer or microbial infection.

Typically, for a quantitative diagnostic assay, a positive result indicating the patient tested has cancer or an infectious disease, is one in which the cells, tissues, or bodily fluids has an PILR expression level at least two times higher, five times higher, ten times higher, fifteen times higher, twenty times higher, twenty-five times higher.

Assay techniques that may be used to determine levels of gene and protein expression, such as PILR, of the present inventions, in a sample derived from a host are well known to those of skill in the art. Such assay methods include radioimmunoassays, reverse transcriptase PCR(RT-PCR) assays, quantitative real-time PCR assays, immunohistochemistry assays, in situ hybridization assays, competitive-binding assays, western blot assays, ELISA assays, and flow cytometric assays, for example, two color FACS analysis for M2 versus M1 phenotyping of tumor-associated macrophages (Mantovani et al., (2002) TRENDS in Immunology 23:549-555).

An ELISA assay initially comprises preparing an antibody specific to PILR. In addition, a reporter antibody generally is prepared that binds specifically to PILR. The reporter antibody is attached to a detectable reagent such as radioactive, fluorescent or an enzymatic reagent, for example horseradish peroxidase enzyme or alkaline phosphatase.

To carry out the ELISA, at least one of the PILR-specific antibody is incubated on a solid support, e.g., a polystyrene dish that binds the antibody. Any free protein binding sites on the dish are then covered by incubating with a non-specific protein, such as bovine serum albumin. Next, the sample to be analyzed is incubated in the dish, during which time PILR binds to the specific PILR antibody attached to the polystyrene dish. Unbound sample is washed out with buffer. A reporter antibody specifically directed to PILR and linked to horseradish peroxidase is placed in the dish resulting in binding of the reporter antibody to any monoclonal antibody bound to PILR. Unattached reporter antibody is then washed out. Reagents for peroxidase activity, including a colorimetric substrate are then added to the dish. Immobilized peroxidase, linked to PILR antibodies, produces a colored reaction product. The amount of color developed in a given time period is proportional to the amount of PILR protein present in the sample. Quantitative results typically are obtained by reference to a standard curve.

A competition assay may be employed wherein antibodies specific to PILR are attached to a solid support and labeled PILR and a sample derived from the host are passed over the solid support and the amount of label detected attached to the solid support can be correlated to a quantity of PILR in the sample.

The above tests may be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. The term “blood” is meant to include whole blood, plasma, serum or any derivative of blood.

The broad scope of this invention is best understood with reference to the following examples, which are not intended to limit the inventions to the specific embodiments. The specific embodiments described herein are offered by way of example only, and the invention is to be limited by the terms of the appended claims, along with the full scope of equivalents to which such claims are entitled.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protcols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example 2

### Antibodies

Agonist antibodies against the activating PILRβ and inhibitory PILRα for both human and mouse were generated in-house as described previously (see, e.g., Fournier, et al. supra). Briefly, female Lewis rats were immunized at regular intervals with a fusion protein consisting of the extracellular domain of mouse or human PILRα/β gene fused to the Fc domain of hIg as described previously (see, e.g., Wright, et al. supra). Hybridomas were initially selected that recognized PILRα/β-Ig (but not the control Ig) fusion protein in indirect ELISA. Hybridomas were then further selected based on their ability to recognize neutrophils, PBMCs and appropriate stably transfected mast cell lines. Additionally antibodies were characterized as agonist antibodies based on their ability to degranulate mast cells.

### Example 3

### Generation of PILRβ−/− Mice

We generated a knockout of the mPILRβ gene in mice using homologous recombination in mouse embryonic stem cells and subsequent blastocyst injection of the appropriate targeted ES cells to create the gene targeted mice. The mouse chromosome 5 sequence (n.t. #135,226,000˜135,306,000) was retrieved from the Ensemb1 database Build 30 and used as reference in this project. BAC clone RP23-131D06 was used for generating homologous arms and southern probes by PCR or RED cloning/gap-repair method. The 5′ homologous arm (8.7 kb) was generated by RED cloning/gap repair, and the 3′ homologous arm (2.2 kb) was generated by PCR reaction using proofreading LA Taq DNA polymerase (Takara). They were cloned in FtNwCD or pCR4.0 vector and confirmed by restriction digestion and end-sequencing.

The final vector was obtained by standard molecular cloning methods and comprised the homologous arms, the FRT flanked Neo expression cassette (for positive selection of the ES cells), and a DTA expression cassette (for negative selection of the ES cells). The final vector was confirmed by both restriction digestion and end sequencing analysis. NotI was used for linearizing the final vector for electroporation. 3′ external probes were generated by PCR reaction using proofreading LA Taq DNA polymerase (Takara) and tested on genomic Southern analysis for ES screening. It was cloned in pCR4.0-TOPO backbone and confirmed by sequencing. The final vector was injected in blastocysts to generate the PILRβ−/− mice. PILRβ−/− were generated on a C57BL/6 background (from Taconic). The resulting knockout founder mice were genotyped (FIG. 1B). The resulting mice were tested for the absence of the PILRβ gene by analyzing their genetic background by simple sequence length polymorphism. PCR was done using the Taq PCR Master kit (Qiagen).

### Example 4

### Cell Isolation

For mouse cells, whole blood was obtained from 6-8 wk old mice by cardiac puncture and mixed with five times the volume of lysis buffer (44.5 g ammonium chloride, 5.0 g potassium bicarbonate, 2 mM EDTA, pH 7.3) for 5 minutes to remove the RBCs. The mixture was spun down and the pellet containing the leukocytes was resuspended in an appropriate volume of PBS.

### Example 5

### Phenotypic Characterization of PILRβ−/− Mice

The lack of cell surface expression of PILRβ was confirmed by FACS staining mouse leukocytes purified from 6-8 wk old male or female PILR−/− mice and their corresponding C57BL/6J age-matched WT controls. Cells were purified as described above and incubated with anti-mPILRβ, anti-mPILRα or anti-mPILRα/β monoclonal antibodies for 1 h at 4 deg C. Cells were washed twice in staining buffer and further incubated for 30 minutes with PE-conjugated goat anti-rat secondary antibody. Cells were washed and the cell surface expression of PILRα/β in wt and PILRβ−/− mice was determined by flow cytometric analysis using a FACScalibur™, (BD Biosciences, Mountain View, Calif.). A complete blood count was also obtained for these mice using the Advia system. In order to evaluate the knockdown of the PILRβ gene at the mRNA and protein levels, various organs such as the heart, lung, liver, kidney and spleen were harvested and submitted for RT-PCR analysis and immunohistochemistry, respectively. For a cell differential analysis, cells from the bone marrow and erythrocyte-depleted splenocytes were labeled with PE-conjugated anti-GR-1, anti-ClassII, anti-CD3 and anti-NK1.1; FITC-conjugated anti-CD45, anti-CD11c, anti-CD8 and anti-CD25; APC-conjugated anti-CD11b, anti-B220 and anti-CD4 (all from BD Biosciences).

### Example 6

### Bacterial Strain and Culture

The S. aureus strain ATCC 27271 was used for the mouse lung infections. A 1:50 dilution of an overnight culture was made into fresh tryptic soy broth. The staphylococci were grown with shaking at 37° C. to an optical density at 600 nm of 0.9. A 40 ml aliquot of the culture was sedimented by centrifugation at 3000 rpm for 15 minutes and the staphylococci was resuspended in 10 ml HBSS buffer (1×10⁸ CFU per 25 ul).

### Example 7

### Intranasal Lung Infection

For induction of pulmonary infection, female 7-8 wk old C57BL/6J and PILRβ−/− mice were anesthetized and inoculated with 25 ul of the S. aureus slurry into the left nare, as described by [16]. Animals were held upright for 1 minute post inoculation and then placed into the cage in a supine position for recovery and were observed for 48-72 h. A small percentage of animals routinely succumbed within the first 6 h following infection, likely from additive effects of aspiration and anesthesia and were thus not included in subsequent statistical analyses. In some experiments we also dosed female C57BL/6J 8 wk old mice with 1 mg/mouse of anti-PILRβ, anti-PILRα and rIgG1 isotype control (rat-anti-hIL-4) antibodies either s.c. 24 h prior to infection or i.v. 2 h post infection.

### Example 8

### Bacterial Growth Post Infection

To assess the bacterial burden of infected mice, lungs were harvested at 6, 24 and 48 h post-infection and homogenized. Lung homogenates were plated by 10-fold serial dilutions on tryptic soy agar plates. Colonies were counted after 24 h incubation at 37 deg C. and presented as log₁₀ CFU per lung. A portion of the homogenate was processed with STAT-60 (Tel-Test, Friendswood, Tex., USA) and analyzed by RT-PCR.

### Example 9

### Brochoalveolar Lavage (BAL)

Mice were euthanized and the pulmonary cavities opened. Lungs were lavaged with 1 ml of PBS through a polyethylene tube cannulated into the trachea as previously described [17]. BAL specimens were centrifuged and supernatants were collected to measure cytokine levels. The cell pellets were processed either for cytopsin analyses or RT-PCR analyses.

### Example 10

### Histolopathological Analysis

After opening the pulmonary cavity, blood in the lungs was cleared by perfusion through the right ventricle of the heart with 10 ml of 10% formalin until the lungs became whitish. The lungs were dissected out from each mouse and fixed with 10% Formalin in PBS. Sections from these lung specimens were stained using H&E.

### Example 11

### Immunohistochemical Analysis

Paraffin embedded lung sections from WT and PILRβ−/− infected mice were also processed for immunohistochemical analysis using a rabbit polyclonal antibody against anti-human myeloperoxidase Catalog #A0398 (Dako Corporation, Carpinteria, Calif., used at 1-4000) to measure the leves of MPO and neutrophil and macrophage influx into the infected lungs at 24 h and 48 h post infection. Paraffin embedded tissues were sectioned at 5 μm thickness and floated on distilled water at 45° C. Sections were mounted on chemically charged slides followed by drying at room temperature until opaque and placed in the oven at 57° C. overnight. Sections were deparaffinized according to established procedures and quenched with 3% hydrogen peroxide for 10 minutes. They were then cleared in running water followed by TBS (50 mM Tris-hydrogen chloride, 150 mM sodium chloride, and 0.05% Tween 20 at pH 7.6). Slides are then heat retrieved with Citrate Buffer at pH 6.1 for 4 minutes at 123° C. using the Biocare Decloaker chamber. Slides were cooled for 15 minutes and followed by a running tap water rinse.

Slides were then rinsed with Tris Buffered Saline (TBS) for 5 minutes and mounted in the DAKO Autostainer. Slides were covered with fresh TBS to prevent drying of sections during mounting. The sections were incubated with the primary antibody at room temperature for 60 minutes. Followed by 30-minute incubation in Rabbit Envision-Plus Catalog #K4011 (Dako Corporation, Carpinteria, Calif.). Slides were developed with DAB-Plus (Dako Corporation, Carpinteria, Calif.) for 10 minutes, rinsed in running distilled water, counterstained in Modified Mayer's Hematoxylin, blued in 0.3% ammonia water followed by a tap water rinse. Slides were mounted in a routine manner and viewed with a light microscope.

### Example 12

### Preparation of Lung Cell Suspension

After opening the pulmonary cavity of euthanized mice, an 18-gauge needle was used to cannulate the trachea and 1 ml of PBS was slowly injected into the lungs and then withdrawn to collect the BAL fluid. The lungs were then dissected and perfused as described above. Thereafter they were aseptically removed for lung leukocyte isolation as described previously [18]. Briefly, the isolated lung was shred into several small pieces and incubated with 15 ml RPMI containing 250 ug/ml of liberase R1 purified enzyme blend and 100 ug/ml penicillin-streptomycin at 37 deg C. for 1 hour. The enzymatic reaction was stopped by adding 10 ml of ice cold PBS-EDTA and the tissue suspension was incubated on ice for an additional 10 minutes. The digested lungs were further disrupted by pipetting the mixture through a 10 ml pipette several times and then gently pushing the tissue suspension through a nylon screen. The single cell suspension was then washed and centrifuged at 1300 rpm. Contaminating RBCs were lysed by incubating the cell pellet for 5 minutes at room temperature in Red Blood Cell Lysis buffer (Sigma). Cells were finally washed with cRPMI and resuspended in 2 ml of cRPMI and total cell counts were obtained using the Vi-cell Coulter counter.

To determine the cell differential, the single cell suspensions obtained from the lungs of infected wt and PILRβ−/− mice by enzymatic digestion were washed and incubated in staining buffer (PBS containing 2% FBS, 0.1% sodium azide and 2 mM EDTA) containing Fc block CD32/CD16 (clone 2.4G2). Cells were stained for 1 h at 4° C. with directly conjugated mAbs. Cells from wt and PILRβ−/− mice were divided into 5 staining groups and mAbs specific for the different cell types as indicated below were used i.e. 1. Isotype-FITC, Isotype-PE, Isotype-APC; 2. Gr-1-APC, CD11b-FITC, CD11c-PE; 3. F4/80-PE, CD11c-APC, CD11b-FITC; 4. CD3e-FITC, CD4-APC, CD8-PE; 5. CD45-APC, CD3e-FITC, NK1.1-PE.Cell acquisition was performed using FACSCalibur™ (BD Biosciences, Mountain View, Calif.) and the data were analyzed using CellQuest™ software (BD Biosciences).

### Example 13

### Myeloperoxidase Assay

In order to determine the levels of myeloperoxidase in the lungs of infected mice, whole lungs were harvested as indicated above at 6 and 24 h post infection and weighed. The lung tissues were homogenized in 1 ml of PBS and centrifuged at 10000 rpm for 10 minutes at 4° C. After aspirating the supernatant the pellet was resuspended in 1 ml of CTAB followed by the addition of 100 ul of 0.5% sodium deoxycholate. The samples were mixed thoroughly and incubated on ice for 30 minutes. The samples were then centrifuged at 10,000 rpm for 30 minutes at 4°. The supernatant was collected and incubated at 60° C. for 2 h. To measure MPO levels, 50 ul of the supernatant was mixed with 90 ul of TNB solution followed by the addition of 90 ul of stop solution. A MPO standard (20 ug/ml) was appropriately diluted and used as the control. The samples were read using a Plate reader (Molecular Devices) at 450 nm. The absorbance values obtained for each sample were normalized to their respective lung weights and the MPO concentrations were represented as MPO μg/g lung tissue.

### Example 14

### Cytokine Measurement

Infected animals were euthanized at 6, 24 and 48 h post S. aureus infection and a sample of blood was collected by cardiac puncture from these animals and circulating serum cytokine levels were measured. Cytokine levels in the BAL fluid were also determined for these animals at the indicated time points. For all cytokine measurements the mouse Cytokine/Chemokine Milliplex kit was used (Millipore, Billerica, Mass.).

### Example 15

### RNA Expression Analysis

Total RNA was extracted from STAT-60-treated lung homogenates according to the manufacturer's instructions. After isopropyl alcohol precipitation, total RNA was re-extracted with phenol:chloroform:isoamyl alcohol (25:24:1) (Sigma Chemicals) using phase-lock light tubes (Eppendorf).

### Example 16

### Real-Time Quantitative PCR for Gene Expression

DNase-treated total RNA was reverse-transcribed using Superscript II (Invitrogen) according to manufacturer's instructions. Primers were designed using Primer Express (PE Biosystems, Foster City, Calif.), or obtained commercially from Applied Biosystems (Foster City, Calif.). Real-time quantitative PCR on 10 ng of cDNA from each sample was performed using either of two methods. In the first method, two gene-specific unlabelled primers were utilized at 400 nM in a Applied Biosystems SYBR green real-time quantitative PCR assay utilizing an ABI 7000, 7300 or 7900 instrument. In the second method, two unlabelled primers at 900 nM each were used with 250 nM of FAM-labelled probe (Applied Biosystems, Foster City, Calif.) in a TAQMAN™ real-time quantitative PCR reaction on an ABI 7000, 7300 or 7700 sequence detection system. The absence of genomic DNA contamination was confirmed using primers that recognize genomic region of the CD4 promoter. Ubiquitin levels were measured in a separate reaction and used to normalize the data by the Δ-Δ Ct method. Using the mean cycle threshold (Ct) value for ubiquitin and the gene of interest for each sample, the equation 1.8e (Ct ubiquitin minus Ct gene of interest)×10⁴ was used to obtain the normalized values. Measurement of cycle threshold (Ct) values for ubiquitin was also used as a secondary measurement of RNA/cDNA quality and samples were deemed acceptable if they were at a Ct of 23 or less. High quality RNA generally leads to ubiquitin Ct values between 17 and 23 for 10 ng of input cDNA. The absence of genomic DNA contamination was confirmed using primers that recognize a region of genomic DNA. Samples with Ct values for genomic DNA of 35-40 were considered acceptable for analysis.

The Δ-□Δ Ct method described above resulted in normalized expression values relative to the housekeeping gene ubiquitin. Normalized values less than 1.0 were considered to be at the limit of detection for this method and were considered to be negative for analysis. Data sets where at least one value was greater than 1.0 in a particular organ were analyzed statistically. Normalized values were log transformed and analyzed by non-parametric one-way ANOVA Kruskal-Wallis analysis, followed by a Dunn's post-test.

### Example 17

### Statistical Analysis

Data are presented using the mean values (n=5−10) utilizing replicated samples and duplicate or triplicate assays. The statistical significance of mortality studies was determined using the Survival curve test (GraphPad Prism 4.0). The significance of bacterial burden, cytokine production, MPO levels and cell differential counts in the lungs of infected mice was calculated using One-way ANOVA and the two-tailed Student's t test.

### Example 18

### Generation and Characterization of Anti-FDF03/PILR Antibodies

Agonist antibodies against the activating PILRβ and inhibitory PILRα for both human and mouse were generated in-house as described previously (see, e.g., Fournier, et al. supra). Briefly, female Lewis rats were immunized at regular intervals with a fusion protein consisting of the extracellular domain of mouse or human PILRα/β gene fused to the Fc domain of hIg as described previously (Wright et al. (2003) J. Immunol. 171:3034-3046). Hybridomas were initially selected that recognized PILRα/13-Ig (but not the control Ig) fusion protein in indirect ELISA. Hybridomas were then further selected based on their ability to recognize neutrophils, PBMCs and appropriate stably transfected mast cell lines.

Antibodies were further characterized as agonist antibodies specific for murine PILRα (DX276) or PILRβ (DX266) based on their ability to inhibit or activate degranulation (measured by β-hexosaminidase release) in mast cell transfectants expressing PILRα (e.g. DT866) or expressing PILRβ (e.g. DT865), respectively. See Zhang et al. (2004) 173:6786 and Cherwinski et al. (2005) J. Immunol. 174:1348, both of which are hereby incorporated by reference. Briefly, to determine whether an antibody is a PILRβ agonist, degranulation is triggered by incubating 1×10⁶ mouse mast cells with the potential PILRβ agonist antibody for one hour in RPMI 1640 medium in 96-well plates.

To determine whether an antibody was a mouse PILRα agonist, degranulation was triggered by incubating 1×10⁶ mouse mast cells with an agonist antibody that binds to the activating receptor CD200RLa (DX89) for one hour in RPMI 1640 medium in 96-well plates, in the presence and in the absence of the potential PILRα agonist antibody.

For both PILRβ and PILRα agonist assays, a 20 μl sample of supernatant was then mixed with 60 μl of the β-hexosaminidase substrate p-nitrophenol-N-acetyl-β-D-glucosaminide (Sigma-Aldrich, St. Louis, Mo., USA) at 1.3 mg/ml in 0.1 M citric acid, pH 4.5. After 3-4 hours at 37° C., 100 μl of stop solution (0.2 M glycine, 0.2 M NaCl, pH 10.7) was added, and the OD₄₀₅₋₆₅₀ was read using a microplate reader (Molecular Devices, Sunnyvale, Calif., USA). Higher OD₄₀₅₋₆₅₀ reflects more β-hexosaminidase in the supernatant, which in turn reflects enhanced degranulation of the mast cells being assayed. See also U.S. Pat. App. Pub. No. 20030223991.

An antibody that specifically binds to mouse PILRβ and triggers degranulation in mast cell transfectants expressing PILRβ (such as DT865), as measured by β-hexosaminidase release, is an agonistic anti-PILRβ antibody. Such data are and particularly reliable if degranulation is triggered in a concentration-dependent manner.

Similarly, an antibody that specifically binds to PILRα and inhibits degranulation in mast cell transfectants expressing PILRα (such as DT866) that are stimulated with DX87 (an antibody specific for the activating receptor CD200RLa), as measured by β-hexosaminidase release, is an agonistic anti-PILRα antibody. See U.S. Pat. App. Pub. No. 20030223991, the disclosure of which is hereby incorporated by reference in its entirety. Such data are and particularly reliable if degranulation is inhibited in a concentration-dependent manner.

To determine whether an antibody is a mouse PILRβ antagonist, degranulation is triggered by incubating 1×10⁶ mouse mast cells with a ligand for PILRβ, such as murine CD99, for one hour in RPMI 1640 medium in 96-well plates, in the presence and in the absence of the potential PILRβ antagonist antibody. An antibody that specifically binds to PILRβ and inhibits degranulation in mast cell transfectants expressing PILRβ (such as DT865) that are stimulated with CD99, as measured by β-hexosaminidase release, is an antagonistic anti-PILRβ antibody. Such data are and particularly reliable if degranulation is inhibited in a concentration-dependent manner.

One of skill in the art would recognize that the screening assays described in this example for the identification of antagonists of mouse PILRβ and agonists of mouse PILRα could be adapted for identification of antagonists of human PILRβ and agonists of human PILRα. Specifically, antibodies raised to human forms of PILRβ and PILRα could be screened in a mast cell degranulation assays involving human (rather than mouse) mast cells. Human cell lines or animals could be engineered to express the human CD200R1L, PILRβ and/or PILRα for use in screening. Human CD200R1L, also known as CD200RLa, is an activating form of CD200R and is further described at Gene ID No. 344807 at the NCBI website, and the nucleic acid and polypeptide sequences are provided at RefSeq NM_{—}001008784.2 and NP_{—}001008784.2, respectively.

For identification of human PILRα agonists, an agonist antibody specific for the activating human receptor CD200R1L may be used to stimulate degranulation, rather than DX87. Alternatively, an agonist antibody for human PILRβ, previously selected for its ability to stimulate mast cell degranulation, may be used in place of DX87 to stimulate degranulation in human mast cells expressing both expressing both PILRβ and PILRα.

For identification of human PILRβ antagonists, human CD99 (SEQ ID NOs: 6 and 8) is used in place of mouse CD99-like molecule to stimulate degranulation. See, e.g., Shiratori et al. (2004) J. Exp. Med. 199:525 at 532.

A listing of sequence identifiers is provided at Table 12.

TABLE 12
Sequence Identifiers

| SEQ | | |
|---|---|---|
| ID NO: | Description | RefSeq |
| 1 | human PILRα nucleic acid | NM_013439.2 |
| 2 | human PILRα polypeptide | NP_038467.2 |
| 3 | human PILRβ nucleic acid | NM_013440.3 |
| 4 | human PILRβ polypeptide | NP_038468.3 |
| 5 | human CD99 (long isoform) nucleic acid | NM_002414.3 |
| 6 | human CD99 (long isoform) polypeptide | NP_002405.1 |
| 7 | human CD99 (short isoform) nucleic acid | NM_001122898.1 |
| 8 | human CD99 (short isoform) polypeptide | NP_001116370.1 |

1. A method of treating an S. aureus infection comprising administering to a subject in need of such treatment, an effective amount of an antagonist of PILRβ.

2. The method of claim 1 wherein the antagonist of PILRβ is an antibody, antibody fragment, antibody conjugate, a soluble PILRβ □polypeptide, or a soluble PILRβ polypeptide fused to a heterologous protein.

3. The method of claim 2, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

4. The method of claim 1, wherein the antagonist of PILRβ reduces S. aureus infection.

5. The method of claim 1, wherein the S. aureus infection is in at least one lung.

6. The method of claim 1, wherein the antagonist of PILRβ is administered with at least one antibiotic having bateriocidal or bacteriostatic activity against S. aureus.

7. A method of treating an S. aureus infection comprising administering to a subject in need of such treatment, an effective amount of an agonist of PILRα.

8. The method of claim 7 wherein the antagonist of PILRα is an antibody, antibody fragment, or antibody conjugate.

9. The method of claim 8, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

10. The method of claim 7, wherein the agonist of PILRα reduces S. aureus infection.

11. The method of claim 7, wherein the S. aureus infection is in at least one lung.

12. The method of claim 7, wherein the agonist of PILRα is administered with at least one antibiotic having bateriocidal or bacteriostatic activity against S. aureus.

13. A method of prophylactically treating a subject against an S. aureus infection comprising administering to the subject in need of such treatment, an effective amount of an antagonist of PILRβ.

14. The method of claim 13 wherein the antagonist of PILRβ is an antibody, antibody fragment, antibody conjugate, a soluble PILRβ □polypeptide, or a soluble PILRβ polypeptide fused to a heterologous protein.

15. The method of claim 14, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

16-18. (canceled)

19. A method of prophylactically treating a subject against an S. aureus infection comprising administering to the subject in need of such treatment, an effective amount of an agonist of PILRα.

20. The method of claim 19 wherein the agonist of PILRα is an antibody, antibody fragment, or antibody conjugate.

21. The method of claim 20, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

22-24. (canceled)

25. The method of claim 1 wherein the antagonist of PILRβ is a nucleic acid antagonist selected from the group consisting of an antisense nucleic acid or an siRNA.

26. The method of claim 13 wherein the antagonist of PILRβ is a nucleic acid antagonist selected from the group consisting of an antisense nucleic acid or an siRNA.


SEQUENCE LISTING (8 sequences)

SEQ ID NO: 1 - DNA, 1323 bp, Homo sapiens
  sig_peptide (213)..(269)
aataggggaaaataagccagatggataaaggaagtgctggtcaccctggaggtgcactggtttggggaaggctcctggcccccacagccctcttcggagcctgagcccggctctcctcactcacctcaacccccaggcggcccctccacagggcccctctcctgcctggacggctctgctggtctccccgtcccctggagaagaacaaggccatgggtcggcccctgctgctgcccctactgcccttgctgctgccgccagcatttctgcagcctagtggctccacaggatctggtccaagctacctttatggggtcactcaaccaaaacacctctcagcctccatgggtggctctgtggaaatccccttctccttctattacccctgggagttagccacagctcccgacgtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggaccagtctgtgtatttctgccgagttgagctggacacacggagctcagggaggcagcagtggcagtccatcgaggggaccaaactctccatcacccaggctgtcacgaccaccacccagaggcccagcagcatgactaccacctggaggctcagtagcacaaccaccacaaccggcctcagggtcacacagggcaaacgacgctcagactcttggcacataagtctggagactgctgtgggggtggcagtggctgtcactgtgctcggaatcatgattttgggactgatctgcctcctcaggtggaggagaaggaaaggtcagcagcggactaaagccacaaccccagccagggaacccttccaaaacacagaggagccatatgagaatatcaggaatgaaggacaaaatacagatcccaagctaaatcccaaggatgacggcatcgtctatgcttcccttgccctctccagctccacctcacccagagcacctcccagccaccgtcccctcaagagcccccagaacgagaccctgtactctgtcttaaaggcctaaccaatggacagccctctcaagactgaatggtgaggccaggtacagtggcgcacacctgtaatcccagctactctgaagcctgaggcagaatcaagtgagcccaggagttcagggccagctttgataatggagcgagatgccatctctagttaaaaatatatattaacaataaagtaacaaatttaaaaagataaaaaaa

SEQ ID NO: 2 - protein, 303 aa, Homo sapiens
  SIGNAL (1)..(19)
  mat_peptide (20)..(303)
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 3 - DNA, 3632 bp, Homo sapiens
  sig_peptide (2497)..(2553)
agaagggaggtagtcgccctccgtcgtggcctggcgtggattccgagcgttggtgtctggcggtttccgaccgttggtgtctggcacgcgccaccccgatgtaccaggtaaagccctatcacggggtcggcgcccctctccgtgtggagcccacctgcatgtactggctccccaacatgcacggcaggagcggcggcccagcactcggcactggccacttgcagacaagaagacaagaaaatgatttgaggacagcttcaatcgcggtgtgaagaagaaagcaacaaaacgaccactgaaaacaatgccggtggcaaaacatccaaagaaagggtcccaagtggtacatcgtcatagctggaaacagtcagagccaccagccaatgatcttttcaatgctgcgaaagctgccaaaagtgacatgcagtgtggccatgaggtctgccggaagtgacttgttggtgttatctcctgagttaaaatgtgaagggatttttttttttcagattactgagagtcttcagttactagaggcggatttccctgactgaagaccatgttgcaggcccacagctgcctacagaaccgtcccaaaatatggcaaagaaacctattctgagcgatagggtctcaccatgttgcccaggctggtcttgaactcctggactcatcctaaagtgctggcctctcattccctgtctgtgcacacctcacggcaagggccagcctgtttcctcccggtcacctccaaatcttgctgcttttaattcaactcagaggcctagccagggttgagttctcacccacctgtgccgccctgccttgttacctggaagcacagccttggggactgagcaggccctcactgtcactttaagaagggaatcagccactttgtgctcaccacctctggggaaggtgtgagaggagagaaggaagtggctgtttggctgctgacaacatgaagacttcctgcgatgagaacagaggcacaggtgccggccctgcagcccccagaacccggactggagggggccatggggcgccggaccctggccctgccctgggtgctgctgaccctgcgtgtcactgcagggaccccggaggtgtgagtacaagttcggatggaggccaccgagctctcgtccttcaccatccgttgtgggttcctggagtctggctccatctccctggtgactgcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgtcccgccgactcctgcccccattctgcgggcagacctggccgggatcttgggggtctcaggagtccttctctttgactgtggctacctccttcatctgctgtgccgacagaagcaccgccctgcccctaggctccagccatcccacaccagctcctaggcactgagagcacgagcatgggcacccagccaggcctcccaggctgctctccacgtcccttatgccactatcaacaccagctgctgcccagctactttggacacagctcacccccgacagggggccgtcctggtgggcatcactccccacccacaccgcacactggccccagggccctgctgcctgggcctccacatccatccctgcacatggcagctttgtctctgttgagaatggactctacgcttaggctgggcagaggcctccctgcactggtcctggcctcactcttttccctgacccttggggcccagggccatggagggacccttaggagttcaatgagagagaccatgaggccactgggctttccccttcccaggcctcctgggtgccacccccttacgttattcttgggcctctaataagtgtcccacaggtgcctggccaggcccacctgctgcagatgtggtctgtgtgtgtgcatgtgtgggtgtgtgtgggcacaggtgtgagtgtgtgagcaacagtaccccattccagtcgtttcctgctgtgactaagtcagcaacacagttcctctgacatgggccttggctgtgcttctttgggggtgaagagattggggaggaagtctccacccctgggaggcagaagccaggcatagcgcgctggctaggactccagtaccgtgaagggaggcagtgagagcagacatctgtgcctcattcctgatctcaaggggaaagcaagaacaagggaggcttcctcaggatctcgaacctgcggaaggaggaccagtctgtgtacttctgccaagtccagctggacatacagatcagggaggctgtcgtggcagtccatcaaggggacccacctcaccatcacccaggccctcaggcagcccctccacagggcccctctcctgcctggacagctctgctggtctccccgtcccctggagaagaacaaggccatgggtcggcccctgctgctgcccctgctgctcctgctgcagccgccagcatttctgcagcctggtggctccacaggatctggtccaagctacctttatggggtcactcaaccaaaacacctctcagcctccatgggtggctctgtggaaatccccttctccttctattacccctgggagttagccatagttcccaacgtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggaccagtctgtgtatttctgccgagtcgagctggacacccggagatcagggaggcagcagttgcagtccatcaaggggaccaaactcaccatcacccaggctgtcacaaccaccaccacctggaggcccagcagcacaaccaccatagccggcctcagggtcacagaaagcaaagggcactcagaatcatggcacctaagtctggacactgccatcagggttgcattggctgtcgctgtgctcaaaactgtcattttgggactgctgtgcctcctcctcctgtggtggaggagaaggaaaggtagcagggcgccaagcagtgacttctgaccaacagagtgtggggagaagggatgtgtattagccccggaggacgtgatgtgagacccgcttgtgagtcctccacactcgttccccattggcaagatacatggagagcaccctgaggacctttaaaaggcaaagccgcaaggcagaaggaggctgggtccctgaatcaccgactggaggagagttacctacaagagccttcatccaggagcatccacactgcaatgatataggaatgaggtctgaactccactgaattaaaccactggcatttgggggctgtttattatagcagtgcaaagagttcctttatcctccccaaggatggaaaaatacaatttattttgcttaccatacaccccttttctcctcgtccacattttccaatctgtatggtggctgtcttctatggcagaaggttttggggaataaatagcgtgaaatgctgctga

SEQ ID NO: 4 - protein, 227 aa, Homo sapiens
  SIGNAL (1)..(19)
  mat_peptide (20)..(227)
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 5 - DNA, 1255 bp, Homo sapiens
  sig_peptide (175)..(240)
ggaggccggggcggggcgggcgcagccggcgctgagcttgcagggccgctcccctcacccgcccccttcgagtccccgggcttcgccccacccggcccgtgggggagtatctgtcctgccgccttcgcccacgccctgcactccgggaccgtccctgcgcgctctgggcgcaccatggcccgcggggctgcgctggcgctgctgctcttcggcctgctgggtgttctggtcgccgccccggatggtggtttcgatttatccgatgcccttcctgacaatgaaaacaagaaacccactgcaatccccaagaaacccagtgctggggatgactttgacttaggagatgctgttgttgatggagaaaatgacgacccacgaccaccgaacccacccaaaccgatgccaaatccaaaccccaaccaccctagttcctccggtagcttttcagatgctgaccttgcggatggcgtttcaggtggagaaggaaaaggaggcagtgatggtggaggcagccacaggaaagaaggggaagaggccgacgccccaggcgtgatccccgggattgtgggggctgtcgtggtcgccgtggctggagccatctctagcttcattgcttaccagaaaaagaagctatgcttcaaagaaaatgcagaacaaggggaggtggacatggagagccaccggaatgccaacgcagagccagctgttcagcgtactcttttagagaaatagaagattgtcggcagaaacagcccaggcgttggcagcagggttagaacagctgcctgaggctcctccctgaaggacacctgcctgagagcagagatggaggccttctgttcacggcggattctttgttttaatcttgcgatgtgctttgcttgttgctgggcggatgatgtttactaacgatgaattttacatccaaagggggataggcacttggacccccattctccaaggcccgggggggcggtttcccatgggatgtgaaaggctggccattattaagtccctgtaactcaaatgtcaaccccaccgaggcacccccccgtcccccagaatcttggctgtttacaaatcacgtgtccatcgagcacgtctgaaacccctggtagccccgacttctttttaattaaaataaggtaagcccttcaatttgtttcttcaatatttctttcatttgtagggatatttgtttttcatatcagactaataaaaagaaattagaaaccaaaaaaaaaaaaaaaaaaaa

SEQ ID NO: 6 - protein, 185 aa, Homo sapiens
  SIGNAL (1)..(22)
  mat_peptide (23)..(185)
MARGAALALLLFGLLGVLVAAPDGGFDLSDALPDNENKKPTAIPKKPSAGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

SEQ ID NO: 7 - DNA, 1207 bp, Homo sapiens
  sig_peptide (175)..(240)
ggaggccggggcggggcgggcgcagccggcgctgagcttgcagggccgctcccctcacccgcccccttcgagtccccgggcttcgccccacccggcccgtgggggagtatctgtcctgccgccttcgcccacgccctgcactccgggaccgtccctgcgcgctctgggcgcaccatggcccgcggggctgcgctggcgctgctgctcttcggcctgctgggtgttctggtcgccgccccggatggtggtttcgatttatccgatgcccttcctggggatgactttgacttaggagatgctgttgttgatggagaaaatgacgacccacgaccaccgaacccacccaaaccgatgccaaatccaaaccccaaccaccctagttcctccggtagcttttcagatgctgaccttgcggatggcgtttcaggtggagaaggaaaaggaggcagtgatggtggaggcagccacaggaaagaaggggaagaggccgacgccccaggcgtgatccccgggattgtgggggctgtcgtggtcgccgtggctggagccatctctagcttcattgcttaccagaaaaagaagctatgcttcaaagaaaatgcagaacaaggggaggtggacatggagagccaccggaatgccaacgcagagccagctgttcagcgtactcttttagagaaatagaagattgtcggcagaaacagcccaggcgttggcagcagggttagaacagctgcctgaggctcctccctgaaggacacctgcctgagagcagagatggaggccttctgttcacggcggattctttgttttaatcttgcgatgtgctttgcttgttgctgggcggatgatgtttactaacgatgaattttacatccaaagggggataggcacttggacccccattctccaaggcccgggggggcggtttcccatgggatgtgaaaggctggccattattaagtccctgtaactcaaatgtcaaccccaccgaggcacccccccgtcccccagaatcttggctgtttacaaatcacgtgtccatcgagcacgtctgaaacccctggtagccccgacttctttttaattaaaataaggtaagcccttcaatttgtttcttcaatatttctttcatttgtagggatatttgtttttcatatcagactaataaaaagaaattagaaaccaaaaaaaaaaaaaaaaaaaa

SEQ ID NO: 8 - protein, 169 aa, Homo sapiens
  SIGNAL (1)..(22)
  mat_peptide (23)..(169)
MARGAALALLLFGLLGVLVAAPDGGFDLSDALPGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK